(19) United States
(12) Patent Application Publication
(10) Pub. No.: US 2010/0021974 A1
(43) Pub. Date: Jan. 28, 2010

(54) Method for Preparation of cRNA

(76) Inventors: Hideji Tajima, Chiba (JP); Masaaki Takahashi, Chiba (JP); Tomoyuki Hatano, Chiba (JP); Hisahiro Tajima, Chiba (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/096,176

(22) PCT Filed: Dec. 5, 2006
(86) PCT No.: PCT/JP2006/324280

§ 371 Date: Jun. 24, 2009

(30) Foreign Application Priority Data
Dec. 5, 2005 (JP) 2005-350949

(51) Int. Cl. C12P 19/34

(52) U.S. Cl. 435/91.53

(57) ABSTRACT

It is an object of the present invention to provide a method for preparing a cRNA, the method being capable of preventing decrease in cRNA yield. In order to achieve the object, the method comprises (a) a step of performing a reaction for preparing a single-stranded cDNA by treating with RNaseH an mRNA-cDNA hybrid prepared by reverse transcription and a reaction for preparing a double-stranded cDNA from the single-stranded cDNA and then inactivating the RNaseH contained in the resultant reaction solution; (b) a step of contacting the reaction solution with a solid support having a cationic group on its surface under pH conditions where the cationic group is positively charged; (c) a step of separating the solid support from the reaction solution; (d) a step of eluting the double-stranded cDNA from the solid support; and (e) a step of performing a transcription reaction for preparing a cRNA from the double-stranded cDNA.

### TECHNICAL FIELD

The present invention relates to a method for preparing cRNA.

### BACKGROUND ART

Analysis using DNA arrays (such as gene expression analysis) is performed, for example, by contacting a sample nucleic acid to be analyzed with a DNA array and then detecting the presence or absence of hybridization of the sample nucleic acid. Gene expression analysis aims at quantitatively determining mRNA contained in total RNA. When the sample nucleic acid is prepared by amplification methods such as RT-PCR or PCR, there is a possibility that, depending on primer designing, the amount of the prepared sample nucleic acid does not reflect the amount of mRNA that is a template (i.e., accurate quantitative determination is compromised). Besides, since mRNA is very unstable and easily disrupted, sufficient amount of sample nucleic acid may not be obtained when an amplification method accompanied with extreme temperature change is used for preparation.

As a method which dissolves these problems, a reverse transcription using oligo d(T)24 primers containing T7 RNA polymerase promoter sequence has been developed. Preparation of Sample Nucleic Acid Using this Reverse Transcription is Performed as Described below. Briefly, total RNA containing mRNA is extracted from a biological material or the like, then, an mRNA-cDNA hybrid is prepared by reverse transcription. Subsequently, the mRNA-cDNA hybrid is treated with RNaseH (ribonuclease H) to thereby prepare a single-stranded cDNA, from which a double-stranded cDNA is prepared. Then, a cRNA is prepared from the double-stranded cDNA by in vitro transcription. The thus prepared cRNA retains higher quantitative determination property than those sample nucleic acids prepared by amplification methods such as RT-PCR or PCR, reflecting the amount of intracellular mRNA. Therefore, such cRNA improves the accuracy in gene expression analysis.

On the other hand, Charge Switch Technology (CST, registered trademark) is known as a technique for purifying nucleic acids (Patent Documents 1 and 2, Non-Patent Document 1). In CST, a solid support having a cationic group on its surface (e.g., magnetic particles) is contacted with a nucleic acid under acidic conditions. When such a solid support is contacted with a nucleic acid under acidic conditions, the cationic group is positively charged and the nucleic acid negatively charged is electrostatically bound to the cationic group. By adjusting the salt concentration when contacting a solid support with a nucleic acid, it is possible to adjust the size of the nucleic acid capable of binding to the positively charged cationic group. Therefore, it is possible to isolate the nucleic acid of a desired size by separating the solid support. The nucleic acid electrostatically bound to the positively charged cationic group may be eluted by neutralizing the electric charge of the cationic group with alkali.

### Patent Document 1: Japanese Unexamined Patent Publication/PCT No. 2004-501054

### Patent Document 2: WO 99/29703

Non-Patent Document 1: Charge Switch PCR Glean-Up Kit; Catalogue No. CS12000; Invitrogen

### DISCLOSURE OF THE INVENTION

### Problem for Solution by the Invention

The present inventors have found that when RNaseH remains undenatured or uninactivated in a reaction solution resulting from a reaction for preparing single-stranded cDNA by treating mRNA-cDNA hybrid obtained by reverse transcription with RNaseH and a reaction for preparing double-stranded cDNA from the single-stranded cDNA (e.g., RNaseH remains undenatured or uninactivated in the reaction solution when organic solvents, protein denaturants or the like are not contained in the reaction solution), the RNaseH binds to a solid support when the double-stranded cDNA is isolated from the reaction solution using the solid support having a cationic group on its surface, and mixes with the isolated double-stranded cDNA; as a result, in the preparation of cRNA from the double-stranded cDNA, cRNA is degraded by the RNaseH and thus the cRNA yield decreases.

Under circumstances, it is an object of the present invention to provide a method of preparing cRNA from double-stranded cDNA, comprising performing a reaction preparing single-stranded cDNA by treating with RNaseH mRNA-cDNA hybrid obtained by reverse transcription and a reaction for preparing double-stranded cDNA from the single-stranded cDNA and isolating the double-stranded cDNA from the resultant reaction solution using a solid support having a cationic group on its surface, the method being capable of preventing the decrease of cRNA yield.

### Means to Solve the Problem

In order to achieve the above-described object, the cRNA preparation method of the present invention comprises the following steps (a) to (e).

(a) a step of performing a reaction for preparing a single-stranded cDNA by treating with RNaseH an mRNA-cDNA hybrid prepared by reverse transcription and a reaction for preparing a double-stranded cDNA from the single-stranded cDNA and then inactivating the RNaseH contained in the resultant reaction solution;
(b) a step of contacting the reaction solution with a solid support having a cationic group on its surface under pH conditions where the cationic group is positively charged;
(c) a step of separating the solid support from the reaction solution,
(d) a step of eluting the double-stranded cDNA from the solid support; and
(e) a step of performing a transcription reaction for preparing a cRNA from the double-stranded cDNA.

When RNaseH remains undenatured or uninactivated in a reaction solution (e.g., when organic solvents, protein denaturants or the like are not contained in the reaction solution), the RNaseH contained in the reaction solution binds to a solid support when double-stranded cDNA is isolated from the reaction solution using the solid support having a cationic group on its surface, and mixes with the isolated double-stranded cDNA, as a result, in the preparation of cRNA from the double-stranded cDNA, cRNA is degraded by the RNaseH and thus the cRNA yield decreases. In the method of cRNA preparation of the present invention, it is possible to prevent the decrease of cRNA yield caused by RNaseH because the RNaseH contained in the reaction solution is inactivated prior to isolation of the double-stranded cDNA from the reaction solution.

In the method of cRNA preparation of the present invention, it is preferable in step (b) described above that the reaction solution is contacted with the solid support under pH conditions where the cationic group is positively charged and in the presence of ammonium ions. By selecting such conditions, the binding of dNTP to the positively charged cationic group can be prevented. As a result, it is possible to bind the double-stranded cDNA to the positively charged cationic group efficiently.

The cRNA preparation method of the present invention preferably comprises, prior to step (d) described above, a step of washing the solid support separated in step (c) described above in the presence of ammonium ions. By comprising this step, it is possible to detach the dNTP bound to the solid support.

In the cRNA preparation method of the present invention, the solid support is preferably particles. When the solid support is particles, it is possible to disperse the solid support in the reaction solution. Hence it is possible to improve the reactivity between the cationic group present on the surface of the solid support and the double-stranded cDNA.

In the cRNA preparation method of the present invention, the particles are preferably magnetic particles. When the solid support is magnetic particles, it is possible to capture the solid support dispersed in the liquid with a magnet to thereby separate the solid support from the liquid easily. Hence, it is possible to realize the automation of cRNA preparation.

In the cRNA preparation method of the present invention, it is preferable to separate the magnetic particles from the reaction solution using a magnet in step (c) described above. By this operation, it is possible to capture the solid support dispersed in the liquid with a magnet to thereby separate the solid support from the liquid easily. Hence, it is possible to realize the automation of cRNA preparation.

In the cRNA preparation method of the present invention, the cRNA is preferably a sample for microarray analysis. That is the cRNA preparation method of the present invention is suitable for preparing samples for microarray analysis.

### EFFECT OF THE INVENTION

According to the present invention, RNaseH contained in a reaction solution resulting from a reaction for preparing single-stranded cDNA by treating. mRNA-cDNA hybrid obtained by reverse transcription with RNaseH and a reaction for preparing double-stranded cDNA from the single-stranded cDNA is inactivated prior to isolation of the double-stranded cDNA from the reaction solution using a solid support having a cationic group on its surface. Therefore, it is possible to effectively prevent the decrease of cRNA yield caused by the mixing of RNaseH.

### BRIEF DESCRIPTION OF THE DRAWINGS

FIG. 1 is a diagram showing the results of size distribution measurement with Agilent 2100 Bioanalyzer.

FIG. 2 is a diagram showing the results of size distribution measurement with Agilent 2100 Bioanalyzer.

FIG. 3 is a diagram showing the results of size distribution measurement with Agilent 2100 Bioanalyzer.

FIG. 4 is a diagram showing the results of size distribution measurement with Agilent 2100 Bioanalyzer.

FIG. 5 is a diagram showing the results of size distribution measurement with Agilent 2100 Bioanalyzer.

FIG. 6 is a diagram showing the results of size distribution measurement with Agilent 2100 Bioanalyzer.

FIG. 7 is a diagram showing the results of size distribution measurement with Agilent 2100 Bioanalyzer.

FIG. 8 is a diagram showing the results of size distribution measurement with Agilent 2100 Bioanalyzer.

FIG. 9 is a diagram showing the results of measurement of samples with GeneChip probe array.

FIG. 10 is a diagram showing the results of comparison of correlations between sample profiles with scatterplot.

### BEST MODE FOR CARRYING OUT THE INVENTION

### Step (a)

Step (a) is a step of performing a reaction for preparing a single-stranded cDNA by treating with RNaseH an mRNA-cDNA hybrid prepared by reverse transcription and a reaction for preparing a double-stranded cDNA from the single-stranded cDNA and then inactivating the RNaseH contained in the resultant reaction solution.

Reverse transcription is performed using mRNA as a template. mRNA may be prepared from a biological sample, environmental sample or the like by conventional methods. Examples of biological samples include, but are not limited to, whole blood, serum, buffy coat, urine, stool, cerebrospinal fluid, sperm, saliva, tissues (such as cancer tissues or lymph nodes) and cell cultures (such as mammal cell cultures or bacterial cultures). Examples of environmental samples include, but are not limited to, soil, water and air. The organisms from which biological samples are derived are not particularly limited. For example, the biological sample may be derived from animals, plants, yeasts, fungi, bacteria, virus, etc. mRNA may be prepared, for example, by treating a biological sample, environmental sample or the like with guanidine reagent, phenol reagent or the like to thereby obtain total RNA and then subjecting the total RNA to affinity column method using oligo dT-cellulose or poly U-Sepharose containing Sepharose 2B as a carrier, or to batch method.

For performing reverse transcription, first, the conformation of mRNA is degraded. At this time, the reaction temperature is usually 65-75° C., preferably 70° C.; and the reaction time is usually 5-15 min, preferably 10 min.

Synthesis of the 1st strand cDNA by reverse transcription may be performed by conventional methods. At this time, the reaction temperature is usually 37-45° C., preferably 42° C.; and the reaction time is usually 30-120 min, preferably 120 min. Reverse transcription is performed with primers and a reverse transcriptase. The primers used in reverse transcription are not particularly limited as long as they are capable of annealing to the relevant template RNA. For example, primers having a nucleotide sequence complementary to a specific template RNA (specific primers), oligo dT (deoxythymine) primers, primers having a random sequence (random primers) or the like may be used. The length of primers for reverse transcription (e.g., oligo dT primers commonly used in expression analysis) is usually 20- to 50-mer, preferably 40-mer. Preferably, primers are used at a final concentration of approximately 200 pmol (usually 150-250 pmol, preferably 200 pmol). The reverse transcriptase used in reverse transcription is not particularly limited as long as it has activity of cDNA synthesis using RNA as a template. Examples of reverse transcriptases which may be used include, but are not limited to, avian myeloblastosis virus-derived reverse transcriptase (AMV RTase), Moloney murine leukemia virus-derived reverse transcriptase (MMLV RTase) and Rous associated virus 2-derived reverse transcriptase (RAV-2 RTase). Further, DNA polymerases which also have reverse transcription activity [e.g., Thermus bacteria-derived DNA polymerases (Tth DNA polymerase, etc.), thermophilic Bacillus bacteria-derived DNA polymerases (B. st-derived DNA polymerase, B. ca-derived DNA polymerase, etc.)] may also be used. In Examples described later, the most commonly used SuperScript II reverse transcriptase was used.

By treating with RNaseH the mRNA-cDNA hybrid prepared by reverse transcription, mRNA in the mRNA-cDNA hybrid is degraded. Thus, it becomes possible to synthesize the 2nd strand cDNA using the 1st strand cDNA as a template. The 2nd strand cDNA may be synthesized by conventional methods. As a DNA polymerase in the synthesis of the 2nd strand cDNA from the 1st strand cDNA, for example, an E. coli-derived DNA polymerase may be used usually at a concentration of 10 U/μl. At this time, the reaction temperature is usually 14-20° C., preferably 16° C.; and the reaction time is usually 120-150 min, preferably 120 min. Treatment with RNaseH may be performed according to conventional procedures. The concentration of RNaseH may be adjusted appropriately considering the concentration of the mRNA-cDNA hybrid and so forth. Usually, the concentration of RNaseH is 1-5 U/μl, preferably 2 U/μl.

In the reverse transcription and the preparation of double-stranded cDNA from single-stranded cDNA, deoxynucleoside triphosphate (dNTP) is used as a substrate for DNA synthesis. Although the term “dNTP” may mean one of dATP, dTTP, dCTP and dGTP, or a mixture of two or more of them, usually it means a mixture of dATP, dTTP, dCTP and dGTP.

The reaction solution resulting from a reaction for preparing single-stranded cDNA by treating with RNaseH mRNA-cDNA hybrid obtained by reverse transcription and a reaction for preparing double-stranded cDNA from the single-stranded cDNA contains the double-stranded cDNA, reverse transcriptase, DNA polymerase, primers, deoxynucleoside triphosphate (dNTP), RNaseH and so forth. The reaction solution is usually a buffer and does not contain organic solvents or protein denaturants. Therefore, RNaseH remains undenatured or uninactivated in the reaction solution. It should be noted that the term “reaction solution” used herein includes not only the reaction solution immediately after the reaction but also the reaction solution which has been treated/processed variously after the reaction (e.g., concentrated, diluted, purified or the like).

The method for inactivating RNaseH is not particularly limited. For example, the RNaseH contained in the reaction solution may be inactivated by heating the reaction solution usually at 60-70° C., preferably at 65° C., usually for 5-15 min, preferably for 10 min in the presence of a reducing agent such as DTT or TCEP which is usually contained in a commercial reagent for expression analysis. In other words, it is not necessarily required to newly add a reducing agent to the reaction solution for inactivating the RNaseH. The RHaseH may be inactivated utilizing the reducing reagent contained in a commercial reagent for expression analysis.

### Step (b)

Step (b) is a step of contacting the reaction solution with a solid support having a cationic group on its surface under pH conditions where the cationic group is positively charged.

Step (b) is performed after step (a).

The shape, material, etc. of the solid support are not particularly limited, but the solid support is preferably particles, more preferably magnetic particles. Since it is possible to disperse particles in a liquid, use of particles as a solid support may improve the reactivity between the cationic group present on the surface of particles and the double-stranded cDNA. Further, by using magnetic particles as a solid support, it becomes possible to capture the particles dispersed in the liquid with a magnet to thereby separate the particles from the liquid easily. Hence, it is possible to realize automation of cRNA preparation.

The solid support may take various shapes in addition to particles, e.g., flat sheet, stick-like, string-like, tape-like or yarn-like shape. Particles are usually spherical, but they may be in an indefinite form. The particle size is not particularly limited. Usually, the particle size is 0.05-0.1 μm, preferably 0.08 μm.

Examples of materials of the solid support include, but are not limited to, glass, silicone, ceramics, water-insoluble polymers [e.g., synthetic resins including polystyrene resins such as polystyrene, acrylic resins (methacrylic resins) such as polymethyl methacrylate, polyamide resins, polyesters such as polyethylene terephthalate, and polycarbonate, polysaccharides such as agarose, dextran and cellulose; and proteins such as gelatin, collagen and casein] and composite materials thereof. Magnetic particles contain a magnetic substance such as iron hydroxide or iron oxide hydrate.

The “surface” of a solid support where a cationic group is present means any surface capable of contacting a liquid. This term includes not only the outer surface of the solid support but also the inner surface thereof into which a liquid is capable of infiltrating (e.g., the internal surface of pores possessed by the solid support).

A cationic group is a functional group which is capable of being positively charged when pH is shifted to the acid side (usually pH 6.0 or less, preferably pH 5.0) and also capable of becoming electrically neutral when pH is shifted to the neutral or alkaline side (usually pH 7.5 or more, preferably pH 8.5). The type of the cationic group is not particularly limited. Examples of cationic groups include, but are not limited to, amino group; monoalkylamino group such as methylamino group or ethylamino group; dialkylamino group such as dimethylamino group or diethylamino group; imino group; and guanidino group.

As the solid support having a cationic group on its surface, a commercial product (e.g., CST PCR Clean Up Kit, Invitrogen, Cat. No. CS12000) may be used. Alternatively, a solid support may be used which has been prepared by chemically binding a cationic group to the surface thereof by conventional methods.

The reaction solution and the solid support having a cationic group on its surface are contacted with each other under pH conditions where the cationic group is positively charged.

The pH at which a cationic group is positively charged varies depending on the type of the cationic group. Usually, that pH is 6.0 or less, preferably 5.0. pH may be adjusted with a weak acid such as phosphoric acid, acetic acid or citric acid.

By contacting the reaction solution with the solid support having a cationic group on its surface under pH conditions where the cationic group is positively charged, negatively charged single-stranded cDNA and double-stranded cDNA are electrostatically bound to the positively charged cationic group.

When the reaction solution is contacted with the solid support having a cationic group on its surface, it is possible to adjust the size of nucleic acid to be electrostatically bound to the cationic group by adjusting salt concentrations. In other words, by adjusting salt concentrations, it is possible to inhibit the binding of nucleic acid such as primers to the cationic group to thereby make the binding of double-stranded cDNA to the cationic group efficient.

In step (b), it is preferred that the reaction solution be contacted with the solid support having a cationic group on its surface under pH conditions where the cationic group is positively charged and in the presence of ammonium ions. By selecting these conditions, it is possible to prevent the binding of dNTP to the positively charged cationic group. Hence, it is possible to allow efficient binding of double-stranded cDNA to the positively charged cationic group.

Ammonium ions may be present at any time point when the reaction solution is in contact with the solid support, i.e., ammonium ions may be added prior to the contact of the reaction solution and the solid support or may be added while the reaction solution is contacted with the solid support.

As the source of ammonium ions, ammonium acetate, ammonium sulfate, ammonium chloride or the like may be used. The concentration of ammonium ion is not particularly limited and may be adjusted appropriately depending of the concentration of double-stranded cDNA, the concentration of dNTP, etc. The ammonium ion concentration is usually 50-500 mM, preferably 100 mM. When the ammonium ion concentration is less than 50 mM, the binding of dNTP to the positively charged cationic group may not be prevented sufficiently. When the ammonium concentration is more than 500 mM, effect corresponding to the increased concentration may not be expected. When ammonium sulfate is used as the source of ammonium ions, it is preferable to add a substance such as magnesium chloride to the reaction solution in order to remove sulfate ions remaining therein.

### Step (c)

Step (c) is a step of separating the solid support from the reaction solution.

Step (c) is performed after step (b).

Since the double-stranded cDNA has been electrostatically bound to the cationic group in step (b), it is possible to isolate the double-stranded cDNA by separating the solid support from the reaction solution. The separation of the solid support may be performed by conventional methods. When the solid support is magnetic particles, it is possible to efficiently separate the solid support with a magnet.

It is preferable to wash the solid support after the separation. By this washing, it is possible to remove substances other than the double-stranded cDNA which are attaching to the solid support. The washing of the solid support is performed under conditions where the double-stranded cDNA bound to the positively charged cationic group will not be detached. The washing of the solid support may be performed by conventional methods. As the washing solution, an aqueous solution such as sterile water or distilled water whose pH is adjusted to 7.0 or less may be used. When the solid support is magnetic particles, it is possible to efficiently disperse the solid support in the washing solution and capture the solid support therefrom by using a magnet.

After separation of the solid support, it is preferable to wash the solid support in the presence of ammonium ions. By this washing, it is possible to detach the dNTP bound to the solid support.

As the source of ammonium ions, ammonium acetate, ammonium sulfate, ammonium chloride or the like may be used. The concentration of ammonium ion is not particularly limited and may be adjusted appropriately depending of the concentration of double-stranded cDNA, the concentration of dNTP, etc. The ammonium ion concentration is usually 50-500 mM, preferably 100 mM. When ammonium sulfate is used as the source of ammonium ions, it is preferable to wash the solid support in the presence of magnesium ions after washing it in the presence of ammonium ions, in order to remove sulfate ions attaching to the solid support. As the source of magnesium ions, magnesium chloride may be used, for example.

### Step (d)

Step (d) is a step of eluting the double-stranded cDNA from the solid support.

Step (d) is performed after step (c). (However, when the solid support is washed after step (c), step (d) is performed after the washing.)

Elution of the double-stranded cDNA from the solid support may be performed, for example, by treating the solid support with an alkali such as a buffer (e.g., Tris-HCl) whose pH is adjusted to 8.0 or more to thereby changing the cationic group present on the surface of the solid support to electrically neutral.

### Step (e)

Step (e) is a step of performing a transcription reaction for preparing a cRNA from the double-stranded cDNA.

Transcription reaction for preparing a cRNA from the double-stranded cDNA may be performed by conventional methods. For the transcription reaction, an in vitro transcription system may be used, for example. As an example of in vitro transcription reaction, a method may be given in which biotinylated UTP and dNTP mix are added to purified cDNA in a tube; T7 RNA polymerase is added thereto; and transcription reaction is performed in the tube. The reaction temperature of transcription reaction using an in vitro transcription system is usually 35-40° C., preferably 37° C.; and the reaction time is usually 4-16 hours, preferably 14 hours. In this transcription reaction, NTPs such as ATP, UTP, CTP and GTP are used as a substrate for cRNA synthesis.

The thus prepared cRNA may be used as a sample for microarray analysis. For example, the cRNA may be used as a sample for gene expression analysis using microarrays (such as DNA arrays). Analysis using microarrays may be performed, for example, by contacting a cRNA sample to be analyzed with a microarray and detecting the presence or absence of hybridization (e.g., fluorescence) of the cRNA sample.

### EXAMPLES

### Test Example 1

### (1) Preparation of Double-Stranded cDNA

Double-stranded cDNA was prepared using reverse transcription.

Reverse transcription was performed using CodeLink Expressin Bioarray System (CodeLink Expression Bioarray System Kit, 24 reactions: GE Healthcare/Amersham Bioscience Cat. 320012). Operational procedures were according to CodeLink User Guide Rev. 2004-09 ver 1.2.

As poly(A)⁺ RNA (mRNA) necessary for preparation of double-stranded cDNA by reverse transcription, a commonly used, commercially available total RNA (rat liver total RNA, Ambion, Cat. 7910) was used. In general, it is considered that the most appropriate yield of RNA per sample necessary for preparation of double-stranded cDNA by reverse transcription is approximately 1 to 15 μg as calculated for total RNA and 0.2 to 2 μg as calculated for mRNA. In this test, 2 μg of total RNA was prepared and used for preparation of double-stranded cDNA by reverse transcription.

Preparation of the 1st strand cDNA by reverse transcription was performed as follows. To 2 μg of total RNA, T7 Oligo(dT) primer (50 μM, control mRNA derived from bacteria for expression analysis (0.5 pg/μl) and nuclease-free sterile water were added. The resultant solution was thermally treated at 70° C. for 10 min, followed by immediate cooling treatment at 4° C. for 2 to 4 min (preferably 3 min). Subsequently, 1st strand cDNA synthesis buffer, dNTP Mix (5-10 mM, preferably 5 mM), RNase inhibitor and reverse transcriptase (200 U/μl, SuperScript II reverse transcriptase) were added thereto and the resultant solution was incubated at 37 to 50° C. (preferably 42° C.) for 60 to 120 min (preferably 120 min).

Preparation of the 2nd strand cDNA by reverse transcription was performed as follows. To the 1st strand cDNA solution, 2nd strand cDNA synthesis buffer, dNTP Mix (5-10 mM, preferably 5 mM), DNA polymerase mix (10 U/μl), E. coli-derived DNA polymerase (10 U/μl), E. coli-derived ligase (10 U/μl), nuclease-free sterile water and RNaseH (1 to 5 U/sample) were added. The resultant solution was incubated at 15 to 17° C. (preferably 16° C.) for 120 to 150 min (preferably 120 min).

(2) Purification of Double-Stranded cDNA

Purification of double-stranded cDNA prepared by reverse transcription is generally performed by a method in which an organic solvent (phenol:chloroform:isoamyl alcohol=25:24:1) is added to the sample in an equal volume to isolate the nucleic acid into the aqueous layer, followed by washing with ammonium acetate and ethanol and extraction into nuclease-free sterile water. In Method 1, double-stranded cDNA prepared by reverse transcription was purified using QIAquick PCR Purification Kit (QIAGEN, Cat. No. 28104/28106) consulting CodeLink User Guide Rev. 2004-09 ver 1.2. In Method 2, double-stranded cDNA prepared by reverse transcription was purified using a magnetic particle reagent CST PCR Purification Kit (sold by Invitrogen, manufactured by DRI, Cat. CS12000) and an automated nucleic acid extractor Magtration System-12GC (hereinafter, referred to as “12GC”) (Precision System Science, Cat. A1006) in order to achieve simplicity by automation and prevention of contamination among samples.

### [Method 1]

Five volumes of buffer PB was added to the double-stranded cDNA solution prepared by reverse transcription and mixed well by pipetting. The resultant mixture (cDNA/buffer PB solution) was poured into a QIAquick spin column mounted in a 2 ml centrifuge tube included in the Kit. Then, the QIAquick spin column was centrifuged at 10000×g for 60 sec. After the centrifugation, the solution which passed through the QIAquick spin column and remained in the 2 ml centrifuge tube was removed. Then, the QIAquick spin column was re-mounted in the centrifuge tube. Buffer PE (700 μl) was added to the QIAquick spin column, which was then centrifuged at 10000×g for 60 sec. After this centrifugation, the solution which passed through the QIAquick spin column and remained in the 2 ml centrifuge tube was removed. Then, the QIAquick spin column was re-mounted in the centrifuge tube and centrifuged again at 10000×g for 60 sec. After the centrifugation, the QIAquick spin column was mounted in a new 1.5 ml tube. Then, 30 μl of nuclease-free sterile water was added to the membrane in the spin column. The spin column was left stationary for 60 sec and then centrifuged at 10000×g for 60 sec. Subsequently, 30 μl of nuclease-free sterile water was added again to the membrane in the spin column. The spin column was left stationary for 30-60 sec (preferably 60 sec) and then centrifuged at 10000×g for 60 sec to thereby secure 60 μl of double-stranded cDNA solution in the total.

### [Method 2]

Into appointed wells in a reagent cartridge for exclusive use for 12GC, 100 μl of purification buffer (well 1), 20 μl of CST magnetic beads (well 2), 700 μl of wash buffer 1 (100 mM ammonium sulfate) (well 3), 700 μl of wash buffer 2 (50 mM magnesium chloride) (well 4), 700 μl of wash buffer 3 (nuclease-free sterile water) (well 5) and 100 μl of elution buffer (10 mM Tris-HCl, pH 8.5) (well 6) were added, respectively. Then, purification of the double-stranded cDNA prepared by reverse transcription was performed according to the 12GC protocol. cDNA purification steps with 12GC are as described below.

Briefly, 80 μl of the purification buffer contained in well 1 was sucked up into a chip and then discharged into a sample tube containing the double-stranded cDNA solution. After the discharge, suction and discharge with the chip were repeated about 15 times to thereby agitate the solution in the sample tube (hereinafter, similar operation is performed in agitation steps). The solution in the sample tube was sucked up into the chip and then discharged into well 2 containing magnetic beads. After the discharge, agitation was performed 300 times to thereby allow the magnetic beads to adsorb cDNA. The magnetic beads were recovered by sucking up the solution in well 2 into a chip to thereby allow the magnetic beads to be adsorbed onto the inner surface of the chip. Then, the magnetic beads alone were discharged into well 3 containing wash buffer 1 (100 mM ammonium sulfate) and washed by repeating suction and discharge with a chip (number of times of agitation: 80) (hereinafter, similar operation is performed in washing steps). After the washing, the magnetic beads were recovered by sucking up the solution in well 3 into a chip to thereby allow the magnetic beads to be adsorbed onto the inner surface of the chip. Then, the magnetic beads alone were discharged into well 4 containing wash buffer 2 (50 mM magnesium chloride) and washed by repeating suction and discharge with a chip (number of times of agitation: 80). After the washing, the magnetic beads were recovered by sucking up the solution in well 4 into a chip to thereby allow the magnetic beads to be adsorbed onto the inner surface of the chip. Then, the magnetic beads alone were discharged into well 5 containing wash buffer 3 (nuclease-free sterile water) and washed by repeating suction and discharge with a chip (number of times of agitation: 80). After the washing, the magnetic beads were recovered by sucking up the solution in well 5 into a chip to thereby allow the magnetic beads to be adsorbed onto the inner surface of the chip. Into this chip containing the magnetic beads alone, 60 μl of the elution buffer (10 mM Tris-HCl, pH 8.5) in well 6 was sucked up. Then, the magnetic beads were discharged into well 7 together with the elution buffer. Double-stranded cDNA was dissociated from the magnetic beads by repeating suction and discharge with a chip.

(3) Sample Preparation Before cRNA Synthesis

For sample preparation before cRNA synthesis (in vitro transcription: IVT), a method is most widely used in which cDNA is purified so that the amount of cDNA becomes as small as possible, and the resultant cDNA as a whole is mixed with cRNA synthesis reagents. This method was used in the present Example. Usually, cDNA is purified to 10-30 μl. In the present Example, purification was completed when the amount of cDNA reached 60 μl. This purified cDNA was concentrated to approximately 10 μl with a centrifugal concentrator and used in the subsequent step (cRNA synthesis). As the centrifugal concentrator, DNA petit Vac (WAKENYAKU CO., LTD., Model PV1200) was used. Operational conditions were 1800-2000 rpm, 0.06 Mpa and 55° C. Under these conditions, the 60 μl solution was concentrated to approximately 10 μl for about 25 sec.

(4) cRNA Preparation by In Vitro Transcription (IVT)

For cRNA preparation by in vitro transcription, a method is used most commonly in which purified double-stranded cDNA is added to a mixture of transcription buffer (Ambion), rTNP mix (25 mM each of T7 ATP, T7 GTP, T7 CTP and T7 UPT), 100 mM DTT, RNase inhibitor (Ambion), 10 mM biotin-11-UTP and 2500 U/μl T7 RNA polymerase and treated at 37° C. for 4 hr to overnight (about 16 hr) (preferably 14 hr). In the present Example, cRNA was prepared as described below according to the procedures of QIAquick PCR Purification Kit (QIAGEN; Cat. No. 28104/28106) described in CodeLink USER GUIDE Rev. 2004-09 ver. 1.2.

The concentrated double-stranded cDNA was mixed with 10× T7 reaction buffer (4.0 μl), 25 mM T7 ATP (4.0 μl), 25 mM T7 GTP (4.0 μl), 25 mM T7 CTP (4.0 μl), 25 mM T7 UTP (4.0 μl), 10 mM biotin-11-UTP (7.5 μl) (Perkin Elmer Cat. No. NEL543) and 10× T7 enzyme mix (4.0 μl) and reacted at 37° C. for 14 hr.

(5) Purification of cRNA

Purification of cRNA was performed by the following Method 3 or 4.

### [Method 3]

First, nuclease-free sterile water was added to the IVT reaction solution to prepare a 100 μl solution. Buffer RLT (350 μl) was added thereto and mixed well. To the resultant mixture, 250 μl of 100% ethanol was added and mixed well. Then, the total or a half volume of the resultant mixture was added to an RNeasy minispin column, which was centrifuged at about 8000×g for 15-30 sec. After the centrifugation, the solution which passed through the RNeasy minispin column and remained in the 2 ml centrifuge tube was removed. When a half volume of the mixture was added to the RNeasy minispin column, the same step was performed again. Buffer RPE (500 μl) was added to the RNeasy minispin column, which was then centrifuged at 8000×g for 15 sec. After the centrifugation, the solution which passed through the RNeasy minispin column and remained in the 2 ml centrifuge tube was removed. Again, Buffer RPE (500 μl) was added to the RNeasy minispin column, which was then centrifuged at 8000×g for 15 sec. After the centrifugation, the solution which passed through the RNeasy minispin column and remained in the 2 ml centrifuge tube was removed. After the removal, the minispin column was mounted in the centrifuge again and centrifuged 8000×g for 2 min for the purpose of drying. After it was confirmed that the ethanol solution was evaporated sufficiently, 50 μl of nuclease-free sterile water was added to the membrane of the RNeasy minispin column. The minispin column was left stationary for 10 min and then centrifuged at 8000×g for 1 min. Again, 50 μl of nuclease-free sterile water was added to the membrane of the RNeasy minispin column; the minispin column was left stationary for 10 min and then centrifuged at 8000×g for 1 min. Finally, 100 μl of cRNA solution was secured.

### [Method 4]

cRNA synthesized from double-stranded cDNA by in vitro transcription was purified using a magnetic particle reagent MagaZorb RNA Mini-Prep Kit (Cortex: Cat. MB2001, MB2004, MB2008) and an automated nucleic acid extractor Magtration System-12GC (Precision System Science: Cat. A1006).

Briefly, into appointed wells in a reagent cartridge for exclusive use for 12GC, 300 μl of binding buffer (well 1), 40 μl of MagaZorb Reagent (magnetic beads) (well 2), 1000 μl of wash buffer 1 (well 3), 1000 μl of wash buffer 2 (1/2 dilution of wash buffer 1) (well 4) and 100 μl of elution buffer (nuclease-free sterile water) (well 5) were added, respectively. Then, purification of the cRNA synthesized from double-stranded cDNA by in vitro transcription was performed according to the 12GC protocol. cRNA purification steps with 12GC are as described below.

Briefly, 200 μl of the binding buffer contained in well 1 was sucked up into a chip and then discharged into a sample tube containing the cRNA solution. After the discharge, suction and discharge with the chip were repeated about 15 times to thereby agitate the solution in the sample tube. The solution in the sample tube was sucked up into the chip, discharged into well 1 and mixed with the remaining binding buffer in well 1, followed by 15 times of agitation. After the agitation, the total volume of the solution in well 1 was sucked up into the chip and discharged into well 2 containing magnetic beads. Then, 600 times of agitation were performed to thereby allow cRNA to be adsorbed onto magnetic beads. The magnetic beads were recovered by sucking up the solution in well 2 to thereby allow the magnetic beads to be adsorbed onto the internal surface of the chip. Then, the magnetic beads alone were discharged into well 3 containing wash buffer 1 and washed by repeating suction and discharge with a chip (number of times of agitation: 50). After the washing, the magnetic beads were recovered by sucking up the solution in well 3 to thereby allow the magnetic beads to be adsorbed onto the inner surface of the chip. The magnetic beads alone were discharged into well 4 containing wash buffer 2 and washed by repeating suction and discharge with a chip (number of times of agitation: 50). After the washing, the magnetic beads were recovered by sucking up the solution in well 4 to thereby allow the magnetic beads to be adsorbed onto the inner surface of the chip. The magnetic beads alone were discharged into well 5 containing elution buffer and washed by repeating suction and discharge with a chip, to thereby dissociate from the magnetic beads cRNA synthesized from double-stranded cDNA by in vitro transcription.

(6) cRNA Yield

The concentration and yield of purified cRNA were calculated as described below. Briefly, 98 μl of nuclease-free sterile water was added to 2 μl of purified cRNA to make the total volume 100 μl (50-fold dilution). Of this 100 μl solution, 60 μl was added to a glass cell and subjected to measurement of absorbance from 220 nm to 320 nm with a Beckman Coulter spectrophotometer model DU530 Life Science UV/Vis Spectrophotometer (Cat. DU530). When the absorbances at 260 nm and at 280 nm are 0.15 or less, similar operations were performed using a sample of 20-fold dilution. cRNA concentration was calculated as follows from the absorbance at 260 nm. It should be noted that cRNA concentration was calculated by the formula described below after correction of the absorbance at 260 nm with the absorbance at 320 nm.

cRNA concentration(μg/ml)=absorbance at 260 nm×dilution ratio×10/cell optical path length(mm)×40(μg/ml)

When a cell with an optical path length of 10 mm is used and the absorbance at 260 nm is 1, the calculated cRNA concentration is 40 μg/ml. In this calculation, dilution ratio is 50.

cRNA yield was calculated by multiplying the calculated cRNA concentration by the total volume of purified cRNA solution recovered (μl).

The purity of purified cRNA was calculated as follows. Briefly, 98 μl of 100 mM Tris-HCl (pH 7.5) was added to 2 μl of purified cRNA to make the total volume 100 μl (50-fold dilution). Of this 100 μl solution, 60 μl was added to a glass cell and subjected to measurement of absorbance from 220 nm to 320 nm with a Beckman Coulter spectrophotometer model DU530 Life Science U/V is Spectrophotometer (Cat. DU530). When the absorbances at 260 nm and at 280 nm are 0.15 or less, similar operations were performed using a sample of 20-fold dilution. cRNA purity was calculated from the absorbance ratio of 260 nm/280 nm after correction of the absorbance at 260 nm with the absorbance at 320 nm.

The size distribution of purified cRNA was confirmed as follows. The size distribution of purified cRNA was measured by adding a sample to RNA 6000 Nano LabChip Kit (Agilent: Cat. 5065-4476) and analyzing with 2100 Bioanalyzer (Agilent: Cat. G2938C). The cRNA sample necessary for this measurement was prepared according to the procedures described in Reagent Kit Guide RNA 6000 Nano Assay Edition October 2003. Adjustment of the concentration of the cRNA sample was also performed according to the standard described in Reagent Kit Guide RNA 6000 Nano Assay Edition October 2003. The resultant sample was added to RNA 6000 Nano LabChip, which was then mounted in 2100 Bioanalyzer for measurement.

The combined method of Method 2 and Method 4 is hereinafter referred to as the “automated purification method (magnetic beads method)”; and the combined method of Method 1 and Method 3 is hereinafter referred to as the “conventional method (spin column method)”. Positive controls in the automated purification method (magnetic beads method) were cRNA samples which were used in the confirmation of performance of cRNA synthesis reagents and were purified by Method 3. Positive controls in the conventional method (spin column method) were cRNA samples which were used in the confirmation of cRNA synthesis reagents and were purified by Method 4.

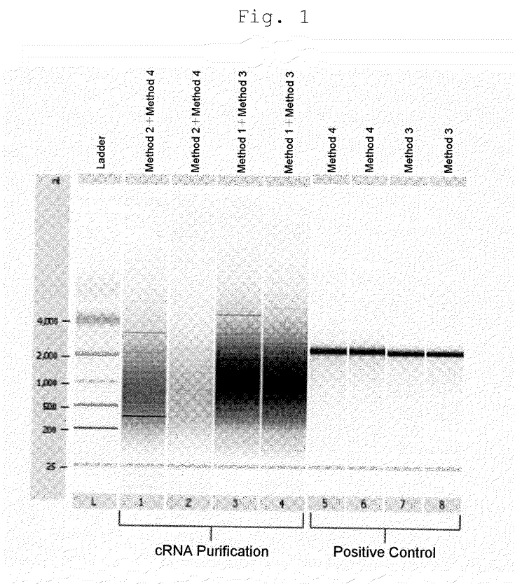

The results concerning the automated purification method (magnetic beads method) are shown in Table 1, and the results concerning the conventional method (spin column method) are shown in Table 2. Further, the results of measurement of size distribution with Agilent 2100 Bioanalyzer are shown in FIG. 1.

TABLE 1

| | Purity | Concentration | Total Yield |
|---|---|---|---|
| Sample No. | (OD 260 nm/280 nm) | (μg/μl) | (μg (100 μl)) |
| 1 | 2.063 | 0.310 | 31.00 |
| 2 | 2.067 | 0.245 | 24.50 |
| Positive Control 1 | 2.057 | 2.146 | 214.6 |
| Positive Control 2 | 2.069 | 2.017 | 201.7 |

TABLE 2

| | Purity | Concentration | Total Yield |
|---|---|---|---|
| Sample No. | (OD 260 nm/280 nm) | (μg/μl) | (μg (100 μl)) |
| 1 | 2.165 | 0.686 | 68.60 |
| 2 | 2.069 | 0.594 | 59.40 |
| Positive Control 1 | 2.070 | 1.665 | 166.5 |
| Positive Control 2 | 2.073 | 1.986 | 198.6 |

As shown in Tables 1 and 2, it could be confirmed that the concentration and yield of purified cRNA in the automated purification method (magnetic beads method) are one half or less of the concentration and yield of purified cRNA in the conventional method (spin column method). In the results of measurement of size distribution with Agilent 2100 Bioanalyzer (FIG. 1), the cRNA concentration in the automated purification method (magnetic beads method) was also greatly inferior to the cRNA concentration in the conventional method (spin column method). However, the Positive Control in the automated purification method (magnetic beads method) and the Positive Control in the conventional method (spin column method) showed almost equal results. Therefore, it was believed that some problem is occurring at the cDNA or cRNA synthesis stage or at the cDNA or cRNA purification stage in the automated purification method (magnetic beads method).

### Test Example 2

The conventional method (spin column method) combining Method 1 and Method 3 was performed together with a modified automated purification method (magnetic beads method) combining Method 1 and Method 4 to measure the purity, concentration, yield and sample distribution of purified cRNA.

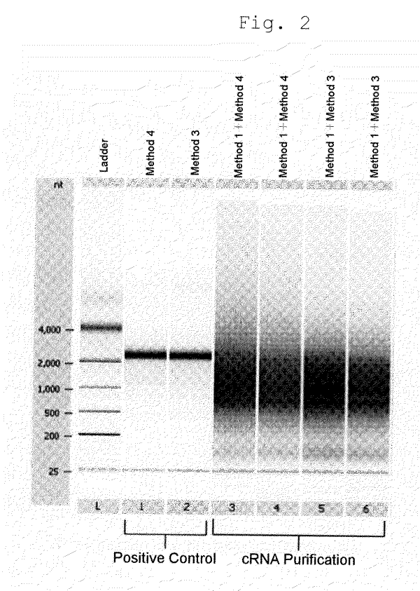

The results concerning the modified automated purification method (magnetic beads method) are shown in Table 3, and the results concerning the conventional method (spin column method) are shown in Table 4. Further, the results of measurement of size distribution with Agilent 2100 Bioanalyzer are shown in FIG. 2.

TABLE 3

| | Purity | Concentration | Total Yield |
|---|---|---|---|
| Sample No. | (OD 260 nm/280 nm) | (μg/μl) | (μg (100 μl)) |
| 1 | 2.040 | 1.490 | 149.0 |
| 2 | 2.037 | 1.270 | 127.0 |
| Positive Control | 2.130 | 2.970 | 297.0 |

TABLE 4

| | Purity | Concentration | Total Yield |
|---|---|---|---|
| Sample No. | (OD 260 nm/280 nm) | (μg/μl) | (μg (100 μl)) |
| 1 | 2.094 | 1.346 | 134.6 |
| 2 | 2.058 | 1.483 | 148.3 |
| Positive Control | 2.067 | 2.318 | 231.8 |

As shown in Tables 3 and 4, no remarkable difference was observed between the modified automated purification method (magnetic beads method) and the conventional method (spin column method). In the results of measurement of size distribution with Agilent 2100 Bioanalyzer (FIG. 2) also, no remarkable difference was observed between the modified automated purification method (magnetic beads method) and the conventional method (spin column method). From these results, it was found that the decrease in cRNA yield in the automated purification method (magnetic beads method) is attributable to a problem which occurred at somewhere after cDNA purification and before the end of in vitro transcription.

### Test Example 3

In view of the results of Test Example 2, the conventional method (spin column method) combining Method 1 and Method 3 was performed together with a modified automated purification method (magnetic beads method) combining Method 2 and Method 3 to measure the purity, concentration, yield and sample distribution of purified cRNA.

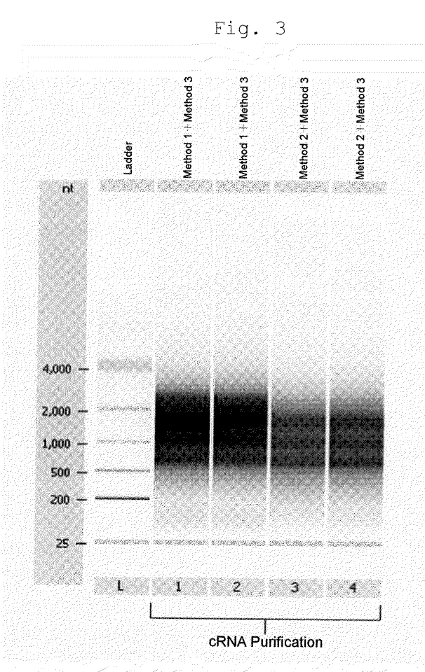

The results concerning the modified automated purification method (magnetic beads method) are shown in Table 5, and the results concerning the conventional method (spin column method) are shown in Table 6. Further, the results of measurement of size distribution with Agilent 2100 Bioanalyzer are shown in FIG. 3.

TABLE 5

| | Purity | Concentration | Total Yield |
|---|---|---|---|
| Sample No. | (OD 260 nm/280 nm) | (μg/μl) | (μg (100 μl)) |
| 1 | 2.165 | 0.650 | 65.00 |
| 2 | 2.130 | 0.545 | 54.50 |

TABLE 6

| | Purity | Concentration | Total Yield |
|---|---|---|---|
| Sample No. | (OD 260 nm/280 nm) | (μg/μl) | (μg (100 μl)) |
| 1 | 2.121 | 1.375 | 137.5 |
| 2 | 2.078 | 1.444 | 144.4 |

As shown in Tables 5 and 6, the concentration and yield of purified cRNA in the modified automated purification method (magnetic beads method) are inferior to the concentration and yield of purified cRNA in the conventional method (spin column method). In the results of measurement of size distribution with Agilent 2100 Bioanalyzer (FIG. 3) also, difference between the two methods is reflected in difference in the darkness. Considering these results together with the results in Test Example 2, it was found that something which inhibits cRNA synthesis exists somewhere between the time of cDNA purification and the time of in vitro transcription. In view of these results, the present inventors have noticed, as the most possible cause, the fact that RNaseH is contained in the reagents used for the synthesis of double-stranded cDNA. While RNaseH is chemically degraded by organic solvents in the conventional method (spin column method), the magnetic particle reagent used in the automated purification method (magnetic beads method) does not contain those components which degrade RNaseH. Further, bad cDNA recovery ratio at the time of cDNA purification is also considered as a cause.

### Test Example 4

In view of the results in Test Example 3, the conventional method (spin column method) combining Method 1 and Method 3 was performed together with a modified automated purification method (magnetic beads method) combining Method 2, Method 1 and Method 3 (double-stranded cDNA is purified by Method 2 and then further purified by Method 1) to measure the purity, concentration, yield and sample distribution of purified cRNA.

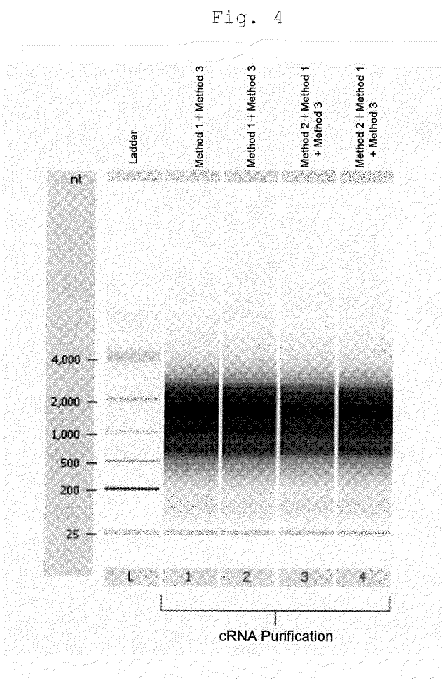

The results concerning the modified automated purification method (magnetic beads method) are shown in Table 7, and the results concerning the conventional method (spin column method) are shown in Table 8. Further, the results of measurement of size distribution with Agilent 2100 Bioanalyzer are shown in FIG. 4.

TABLE 7

| | Purity | Concentration | Total Yield |
|---|---|---|---|
| Sample No. | (OD 260 nm/280 nm) | (μg/μl) | (μg (100 μl)) |
| 1 | 2.118 | 1.480 | 148.0 |
| 2 | 2.066 | 1.489 | 148.9 |

TABLE 8

| | Purity | Concentration | Total Yield |
|---|---|---|---|
| Sample No. | (OD 260 nm/280 nm) | (μg/μl) | (μg (100 μl)) |
| 1 | 2.121 | 1.375 | 137.5 |
| 2 | 2.078 | 1.444 | 144.4 |

As shown in Tables 7 and 8, cRNA yield equivalent to or superior to cRNA yield in the conventional method (spin column method) was obtained in the modified automated purification method (magnetic beads method). In the results of measurement of sample distribution with Agilent 2100 Bioanalyzer (FIG. 4) also, no remarkable difference was observed between the modified automated purification method (magnetic beads method) and the conventional method (spin column method). These results revealed that it is highly possible that the poor cRNA synthesis in in vitro transcription is caused by contamination of RNaseH in the reaction solution.

### Test Example 5

In order to denature RNaseH simply and efficiently, the present inventors have paid their attention to 0.1 M DTT which is combined as a reducing agent in various reagents used in double-stranded cDNA synthesis. When the sample is incubated at 60-70° C. in general (preferably 65° C.) for 10 min, it is possible to denature or inactivate RNaseH with the action of DTT.

The conventional method (spin column method) combining Method 1 and Method 3 was performed together with the automated purification method (magnetic beads method) combining Method 2 and Method 4 to measure the purity, concentration, yield and sample distribution of purified cRNA. In the automated purification method (magnetic beads method), the sample was incubated at 65° C. for 10 min to thereby denature or inactivate the RNase contained in the sample before purification of double-stranded cDNA by Method 2.

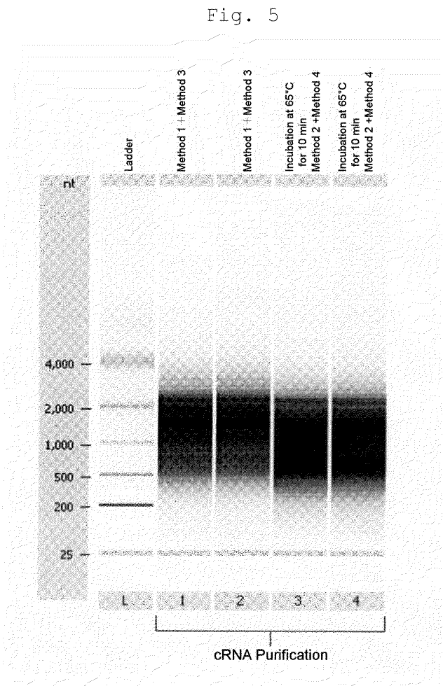

The results concerning the automated purification method (magnetic beads method) are shown in Table 9, and the results concerning the conventional method (spin column method) are shown in Table 10. Further, the results of measurement of size distribution with Agilent 2100 Bioanalyzer are shown in FIG. 5.

TABLE 9

| | Purity | Concentration | Total Yield |
|---|---|---|---|
| Sample No. | (OD 260 nm/280 nm) | (μg/μl) | (μg (100 μl)) |
| 1 | 1.983 | 1.198 | 119.8 |
| 2 | 2.027 | 1.120 | 112.0 |

TABLE 10

| | Purity | Concentration | Total Yield |
|---|---|---|---|
| Sample No. | (OD 260 nm/280 nm) | (μg/μl) | (μg (100 μl)) |
| 1 | 2.010 | 0.819 | 81.90 |
| 2 | 2.016 | 0.836 | 83.60 |

As shown in Tables 9 and 10, cRNA yield superior to the cRNA yield in the conventional method (spin column method) was obtained in the automated purification method (magnetic beads method) by incubating the sample at 65° C. for 10 min before purification of double-stranded cDNA by Method 2. In the results of measurement of size distribution with Agilent 2100 Bioanalyzer (FIG. 5) also, no remarkable difference was observed between the automated purification method (magnetic beads method) and the conventional method (spin column method). These results revealed that it is possible to prepare purified cRNA efficiently in the automated purification method (magnetic beads method) by incubating the sample at 65° C. for 10 min before purification of double-stranded cDNA by Method 2.

### Test Example 6

### (1) cRNA Preparation

The conventional method (spin column method) combining Method 1 and Method 3 and the automated purification method (magnetic beads method) combining Method 2 and Method 4 were performed in accordance with Test Example 1 with necessary modifications. In the automated purification method (magnetic beads method), RNase contained in the sample was denatured or inactivated by incubating the sample at 65° C. for 10 min before purification of double-stranded cDNA by Method 2, in accordance with Test Example 5 with necessary modifications.

As total RNA, human kidney total RNA (Ambion: Cat. 7976) was used instead of rat liver total RNA (Ambion: Cat. 7910) (amount of total RNA used: 2 μg). As an amplification reagent, MessageAmp II Biotin Enhanced (Ambion: Cat. 1791) was used (IVT time: 4 hr).

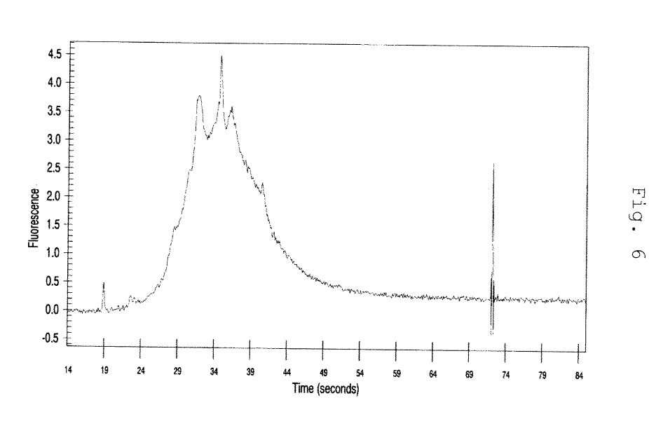

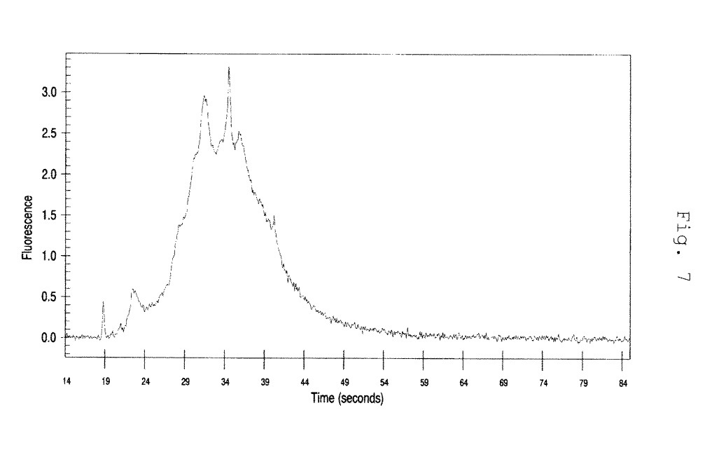

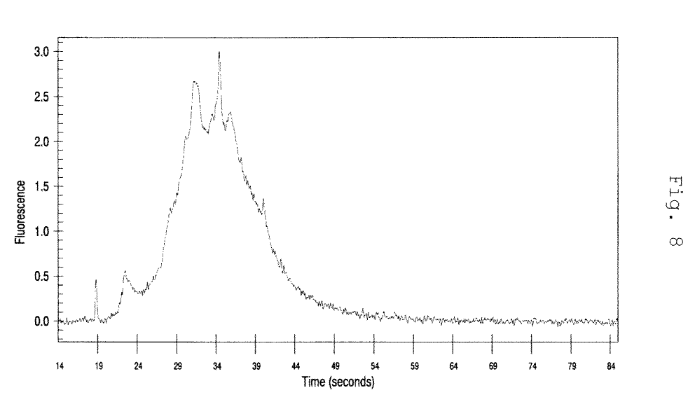

The results of measurement of size distribution with Agilent 2100 Bioanalyzer on sample M purified by the conventional method (spin column method) are shown in FIG. 6, and the results of measurement of size distribution with Agilent 2100 Bioanalyzer on samples A1 and A2 purified by the automated purification method (magnetic beads method) are shown in FIGS. 7 and 8. The yields and purities of samples M, A1 and A2 are shown in Table 11.

TABLE 11

| | Total Yield | Purity |
|---|---|---|
| Sample ID | (μg (100 μl)) | (OD 260 nm/280 nm) |
| M | 27.26 | 2.0 |
| A1 | 46.85 | 1.9 |
| A2 | 44.41 | 1.9 |

### (2) Measurement of Samples with GeneChip Probe Array

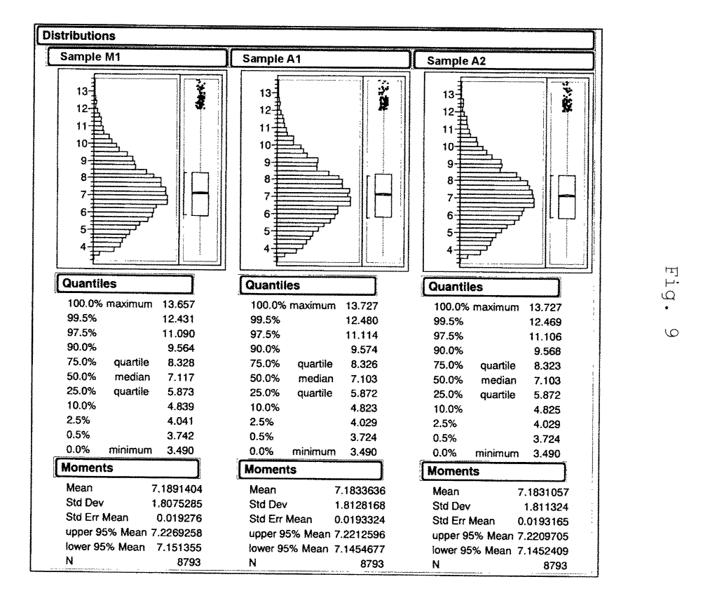

Samples M, A1 and A2 were measured with eukaryotic GeneChip Probe Arrays according to GeneChip Expression Analysis Technical Manual (Affymetrix). Briefly, cDNA samples were cut into approximately 35-200 bp fragments. Then, prehybridization buffer was inserted into eukaryotic GeneChip Probe Arrays, followed by prehybridization. Subsequently, fragmented cRNA samples were inserted into probe arrays, followed by hybridization. Subsequently, cRNA sample-inserted arrays were mounted in Fluidics Station 400/450. The arrays were washed, followed by fluorescence fixation. The resultant signals were read with GeneArray Scanner or Genechip Scanner 3000. The results of measurement are shown in FIG. 9.

In the graphs located on the upper side of FIG. 9, numerical FIGS. 4 to 13 indicate the degree of luminescence. The greater the figure is, the greater the luminescence is. Horizontal bar graphs are showing the distribution of spots on arrays at relevant luminescence degree. It is seen that a large number of spots are converging at luminescence degrees 6 to 8. The tables located on the lower side of FIG. 9 show the results given in the upper side graphs with numerical values. Taking the total number of spots as 100%, ratios of individual luminescence degrees (3.490-13.727) are accumulated. About 65% of the total spot falls within 5.872-8.323 in luminescence degree; 50% of the total spot is 7.103 in luminescence degree.

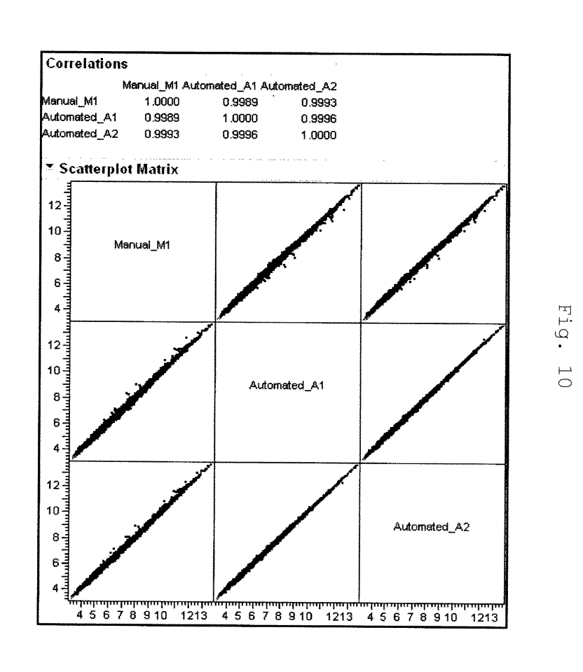

Further, expression profiles of samples M, A1 and A2 were obtained with eukaryotic GeneChip probe arrays. Correlation between profiles was examined by scatterplot on samples M and A1, samples M and A2 and samples A1 and A2. The results are shown in FIG. 10. Respective correlation factors between samples M and A1, samples M and A2 and samples A1 and A2 are shown in Table 12.

TABLE 12

| IDs of Compared Samples | Correlation Factor |
|---|---|
| M vs A1 | 0.9969-0.9989 |
| M vs A2 | 0.9993-0.9993 |
| A1 vs A2 | 0.9996-0.9996 |

1. A method of preparing a cRNA comprising the following steps (a) to (e):
(a) a step of performing a reaction for preparing a single-stranded cDNA by treating with RNaseH an mRNA-cDNA hybrid prepared by reverse transcription and a reaction for preparing a double-stranded cDNA from the single-stranded cDNA and then inactivating the RNaseH contained in the resultant reaction solution;
(b) a step of contacting said reaction solution with a solid support having a cationic group on its surface under pH conditions where the cationic group is positively charged;
(c) a step of separating said solid support from said reaction solution;
(d) a step of eluting said double-stranded cDNA from said solid support; and
(e) a step of performing a transcription reaction for preparing a cRNA from said double-stranded cDNA.

2. The method according to claim 1, wherein said reaction solution is contacted with said solid support under pH conditions where the cationic group is positively charged and in the presence of ammonium ions in step (b).

3. The method according to claim 1 or 2, wherein said method comprises prior to step (d) a step of washing the solid support separated in step (c) in the presence of ammonium ions.

4. The method according to claim 1 or 2, wherein said solid support is particles.

5. The method according to claim 4, wherein said particles are magnetic particles.

6. The method according to claim 5, wherein said magnetic particles are separated from said reaction solution with a magnet in step (c).

7. The method according to claim 1 or 2, wherein said cRNA is a sample for microarray analysis.
